(19) United States
(12) Patent Application Publication
(10) Pub. No.: US 2022/0096692 A1
(43) Pub. Date: Mar. 31, 2022

(54) OZONE, UVC LIGHT AND STEAM SANITIZER WITH ULTRASONIC NEBULIZER

(71) Applicant: EDELBERT GALVÃO DUTRA, FLORIANÓPOLIS - Estado de Santa Catarin (BR)

(72) Inventor: EDELBERT GALVÃO DUTRA, FLORIANÓPOLIS - Estado de Santa Catarin (BR)

(21) Appl. No.: 17/036,842

(22) Filed: Sep. 29, 2020

(51) Int. Cl. A61L 2/24; A61L 2/20; A61L 2/10; A61L 2/07

(52) U.S. Cl. CPC A61L 2/24; A61L 2/202; A61L 2/10; A61L 2202/26; A61L 2202/11; A61L 2202/14; A61L 2202/15; A61L 2/07

(57) ABSTRACT

A device that works with electrical energy through the source (1), being activated by a button with LED (2) that activates the control board (13), responsible for activating the UVC lamp module (14), ozonizer module (15) and the steam generator (8) in their respective operating cycles. The water will be vaporized by the ultrasonic nebulizer, which working together with the ozone generator (10) and the UVC lamp (11) will compose the sanitizing elements distributed by the fans (4) through the grid (5) or the adapter (9) inside the object to be cleaned. The nebulizer consists of the set comprised by the water tank (12), water sensor (3) and steam generator (8) and will only be activated if the sensor (3) detects enough water inside the tank (12). Under the sanitizer are located the feet (6) and the air intake grid (7) for cooling the control board and other components.

### BACKGROUND OF THE INVENTION

### Field of the Invention

Refers to the Utility Model patent for an “Ozone, UVC light and steam sanitizer with ultrasonic nebulizer”, which acts primarily on the cleaning of motoring and motorcycle helmets or other motorized or non-motorized sports, such as cycling helmets, water or snow sports, or even in helmets used by professionals such as Personal Protective Equipment (PPE) from the various areas where helmets accumulate sweat and moisture in the fabric or interior material, in order to eliminate bacteria and microorganisms, eliminating the bad smell and cleaning the inside of the helmet. It also has an adapter that can be used for cleaning shoes, gloves and balaclavas.

### Description of the Related Art

In the state of the art, currently the method used to sanitize helmets, shoes and gloves, consists of the traditional way of cleaning, using chemicals, such as alcohols, detergents and disinfectants, whose residues, if not well removed, can be harmful to the health of the user, in addition to the possibility of damaging the sanitized surface. In addition, the inside of the helmet, shoes and gloves have corners, creases and other places that are difficult to access for cleaning, and may accumulate unwanted substances and bacteria even after traditional cleaning.

Also in the state of the art, steam and its properties are used to facilitate the removal of difficult dirt, but traditional vaporization equipment does not have the ideal ergonomics for use in helmets, shoes and gloves, making it difficult to reach all the points of accumulation of dirt and odors.

Still in the state of the art, there are products for drying or ventilating helmets, shoes and gloves or platforms with fans, but in none of the cases do they wash and dry in one go and with the same efficiency.

It is also known the Patent N.^{o} BR 10 2017 013930 1 of the “DEVICE FOR DRYING, DEODORIZING AND MAINTENANCE OF HELMETS”, owned by the same author/depositor and which in turn consists of a device for drying and deodorizing helmets, which can be coupled directly to the bottom of a bag with handles.

And finally, also owned by the same Applicant, the BR 20 2020 009346 6 of the “DRYER AND SANITIZER FOR HELMETS” is mentioned in the state of the art, which consists of a device with ventilation for drying and eliminating or reducing germs and bacteria and fungi that cause the bad odor of helmets, with the use of ventilation, ozone and ultraviolet light (UVC), but not having the nebulization system and restricted to helmet cleaning only.

### SUMMARY OF THE INVENTION

The present Utility Model aims to present a device with ventilation for drying and eliminating or reducing germs and bacteria and fungi that cause the bad odor of helmets, shoes, gloves or balaclavas with the use of ventilation, ozone, ultraviolet light (UVC) and nebulization of ultrasonic steam. Ozone has a germicidal, bactericidal and anti-virus action, as well as UVC light, which potentiated by the nebulization of ultrasonic steam, promotes sterilization, making a treatment that reduces or eliminates bad odors from the continuous use of helmets, shoes, gloves or balaclava.

A device that works connected to the electrical energy through the source (1), being activated by a button with LED (2) that activates the control board (13), responsible for activating the UVC lamp module (14), ozonizer module (15) and the steam generator (8) in their respective operating cycles. When the cycle starts, the water will be vaporized by the ultrasonic nebulizer, which working together with the ozone generator (10) and the UVC lamp (11) will compose the sanitizing elements distributed by the fans (4) through the grid (5) or the adapter (9) inside the object to be cleaned. The ultrasonic steam nebulizer consists of the set comprised by the water tank (12), water sensor (3) and steam generator (8) and will only be activated if the sensor (3) detects that there is enough water inside the tank (12). Under the sanitizer are located the feet (6) and the air intake grid (7) for cooling the control board and other device components.

### BRIEF DESCRIPTION OF THE DRAWINGS

FIG. 1.1, shows the sanitizer with the grid (5) in top view.

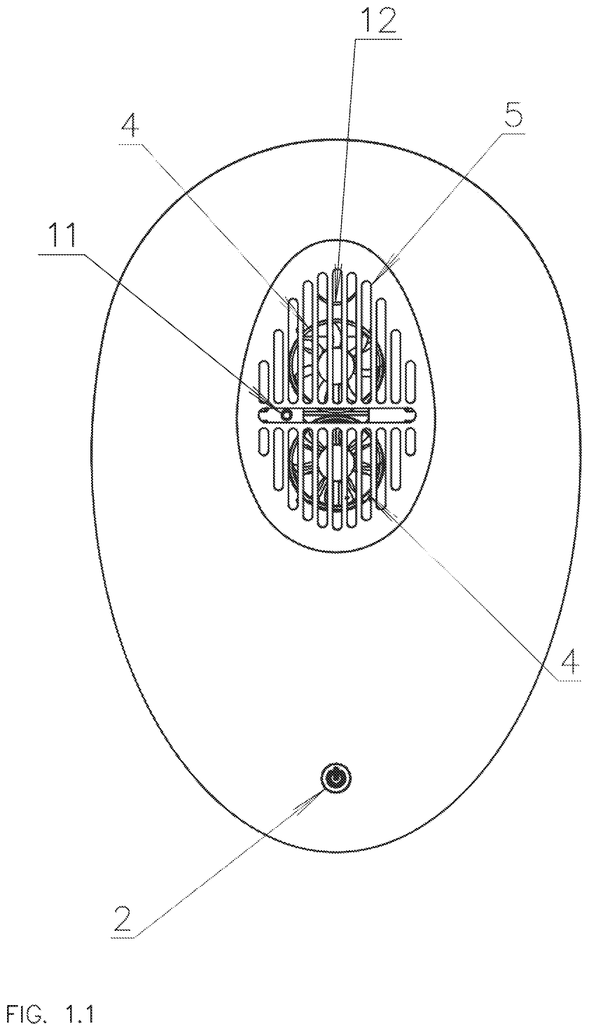

FIG. 1.2, shows the sanitizer in top view without the cover, without the grid (5), and without the fans (4).

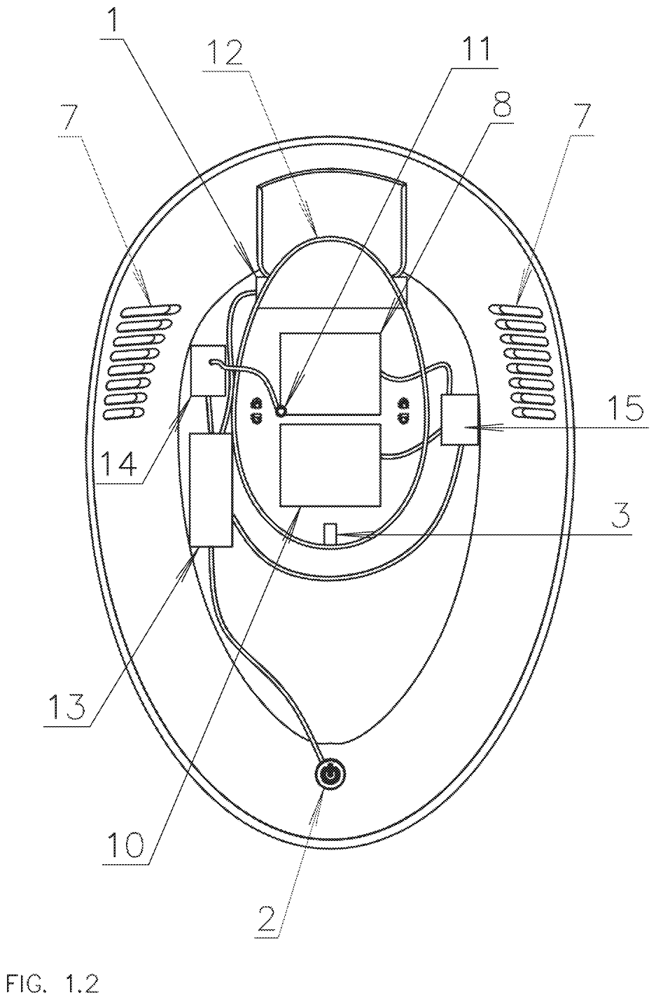

FIG. 1.3, shows the sanitizer with the grid (5) in right-side view.

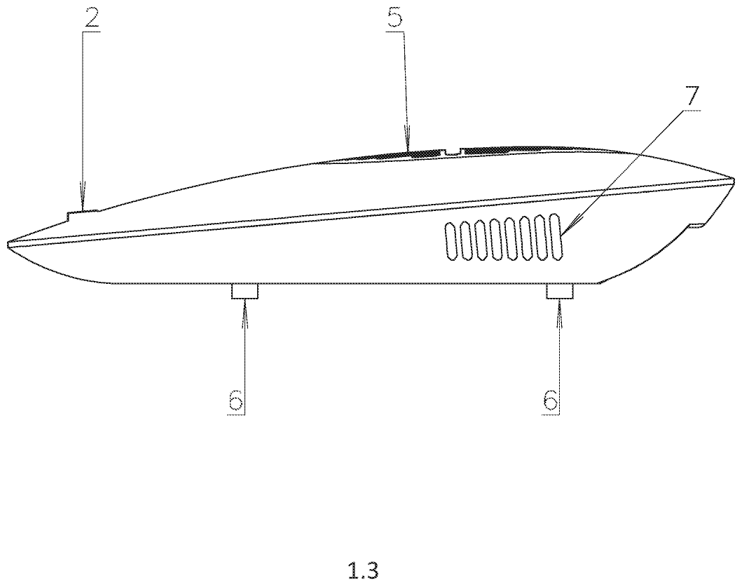

FIG. 1.4, shows the sanitizer with the grid (5) cut in the longitudinal direction seen from the right side.

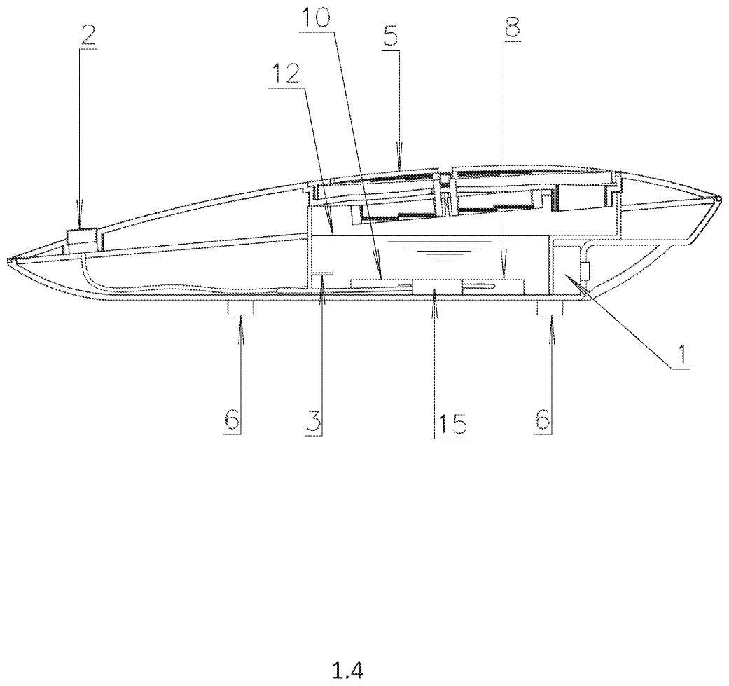

FIG. 1.5, shows the sanitizer with the grid (5) in left-side view.

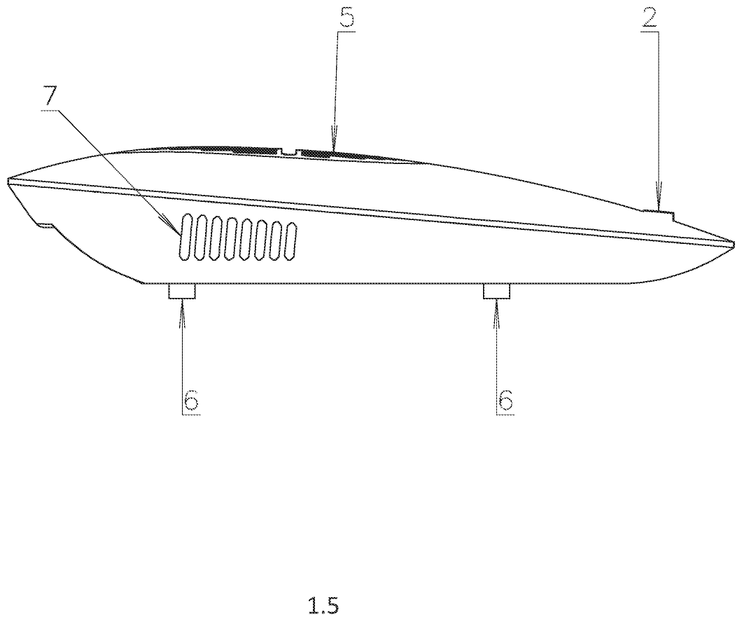

FIG. 1.6, shows the sanitizer with the grid (5) in front view.

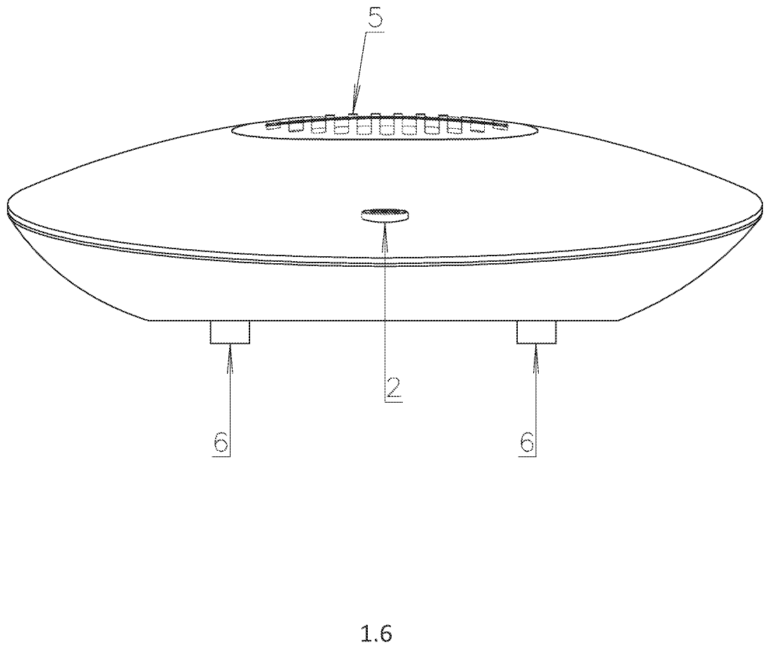

FIG. 1.7, shows the sanitizer with the grid (5) in rear view.

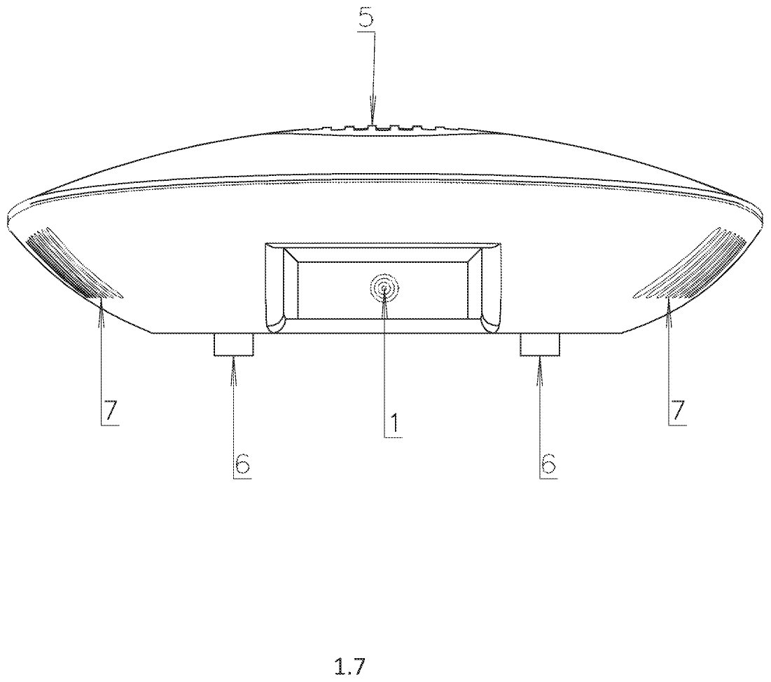

FIG. 1.8, shows the sanitizer with the grid (5) in bottom view.

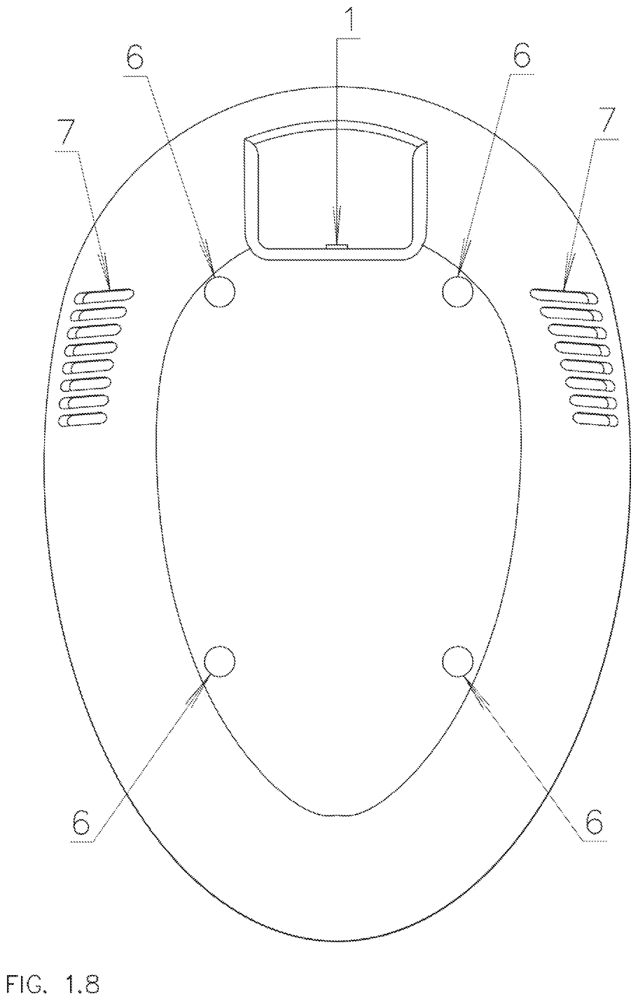

FIG. 1.9, shows the sanitizer with the grid (5) in perspective.

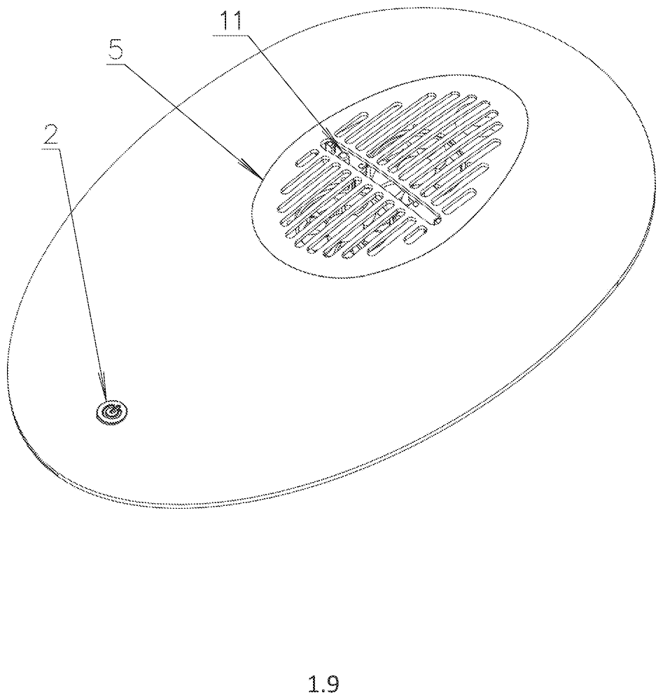

FIG. 1.10, shows the sanitizer without the cover, without the grid (5), and without the fans (4) in perspective.

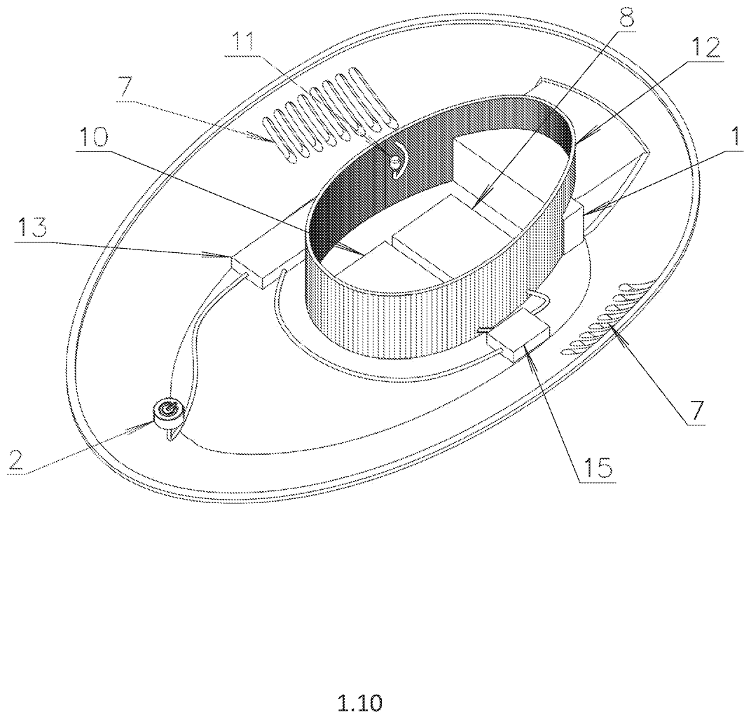

FIG. 2.1, shows the sanitizer with the adapter (9) in top view.

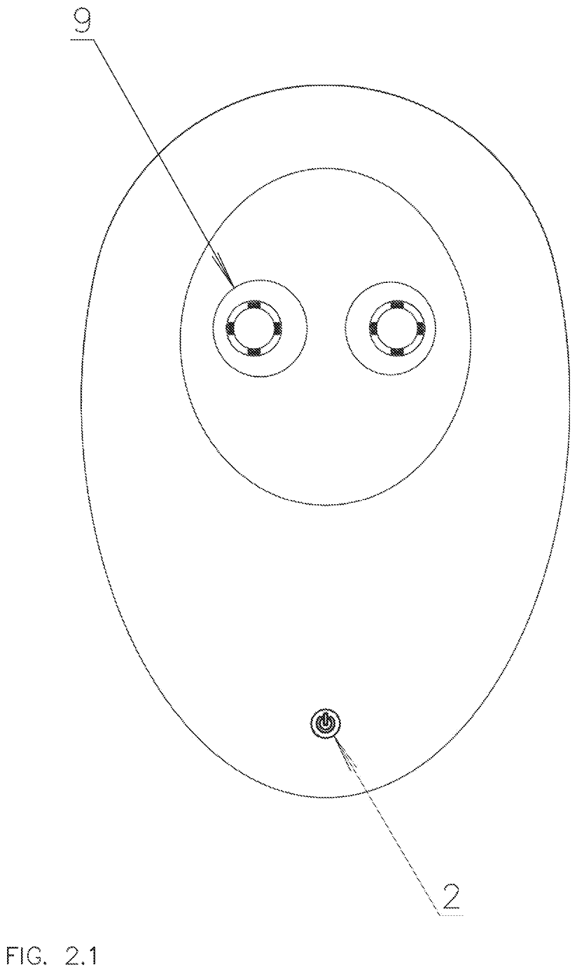

FIG. 2.2, shows the sanitizer with the adapter (9) in right-side view.

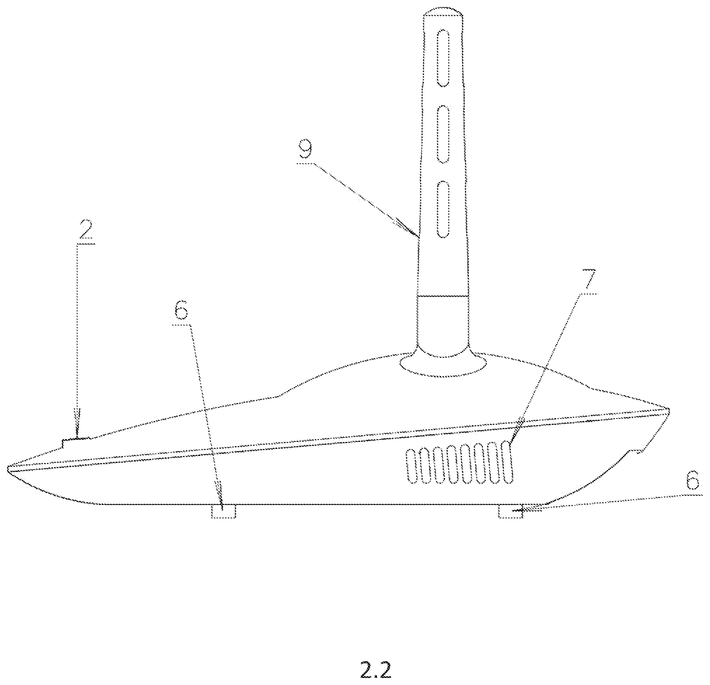

FIG. 2.3, shows the sanitizer with the adapter (9) in left-side view.

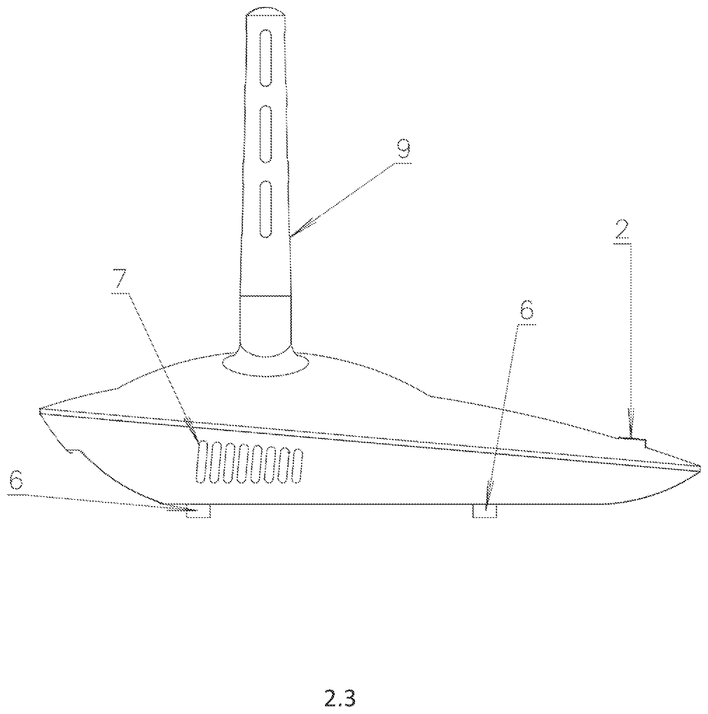

FIG. 2.4, shows the sanitizer with the adapter (9) in front view.

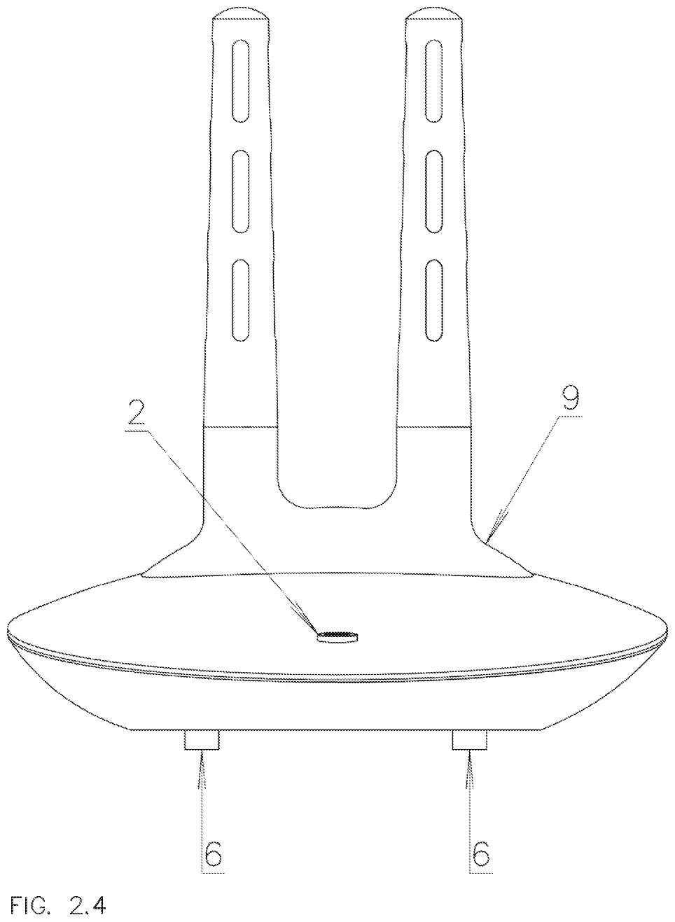

FIG. 2.5, shows the sanitizer with the adapter (9) in rear view.

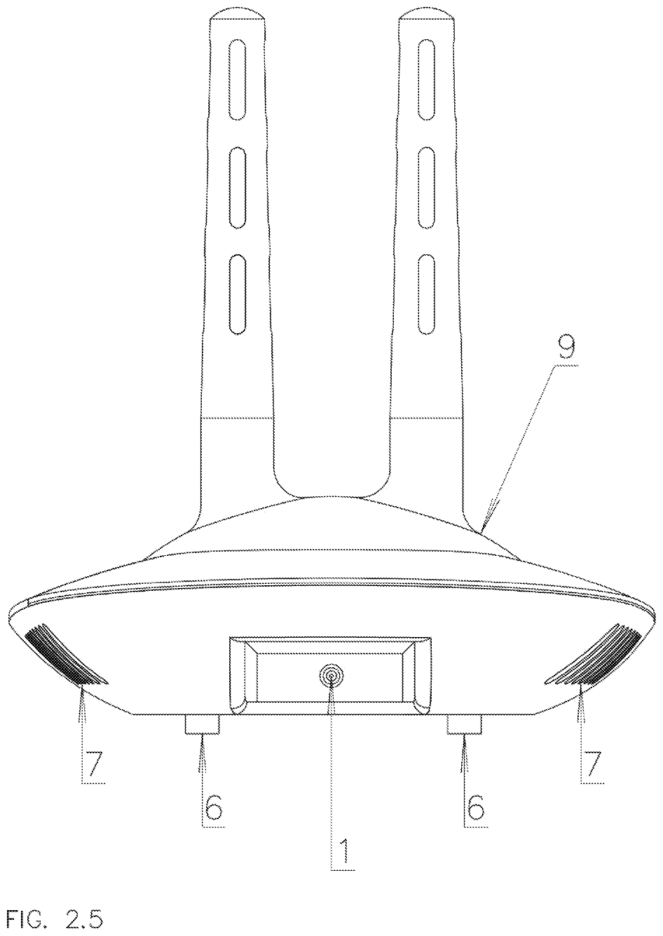

FIG. 2.6, shows the sanitizer with the adapter (9) in perspective.

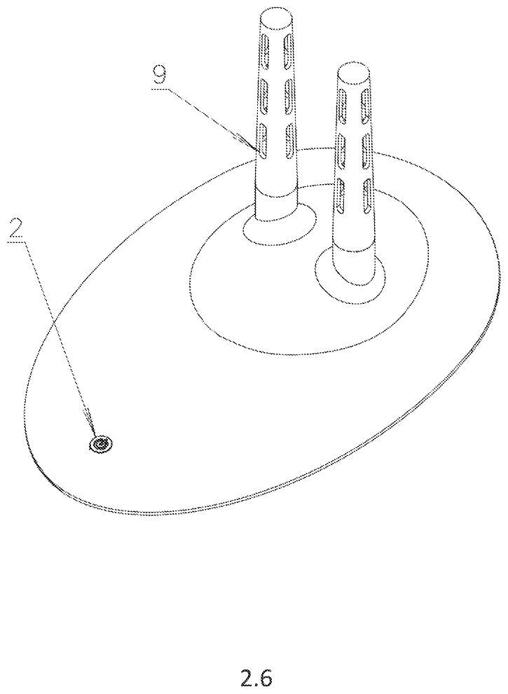

### DETAILED DESCRIPTION OF THE SEVERAL EMBODIMENTS

As indicated in the figures, the present Utility Model, the product consists of a platform on which the object to be cleaned is supported.

With the equipment connected to the power grid through the source (1), it must be activated by the button with LED (2), to start the complete cycle of operation. At the same time, it will start operating the ozone generator (10), the UVC light lamp (11) and the ultrasonic steam nebulizer, whose water must be supplied in the water compartment (12), which in turn has a sensor (3) that will only allow the nebulizer to be activated when it contains water inside. The set formed by the water compartment (12), water sensor (3) and steam generator (8) comprises the nebulizer, while on the platform there are two fans (4) that disperse the sanitizing elements through the grid (5) or the adapter (9) and are responsible for the circulation of air inside the object to be cleaned. The entire system will start operating with the fans (4) turned on at a reduced speed, from 30% to 50%, enough to spread ozonized water steam inside the helmet. Finally, the entire system will shut down automatically. The operating time of a cycle is from 90 to 120 minutes and, if necessary, the cycle can be repeated.

The control board (13) is responsible for managing the components of the set, sending the necessary commands to the UVC light module (14), to the ozonizer module (15) and to the nebulizer so that the activation of the UVC lamp (11) of the ozone generator (10) and the steam generator (17) is started and stopped at the correct intervals.

At the bottom of the sanitizer are located the feet (6) and the air intake grid (7) for ventilation of the control board (13).

The platform is designed to accommodate one helmet, but by removing the grid (5) and fitting the adapter (9) it is possible to sanitize several other types of objects, such as shoes, gloves, balaclavas or any other equipment that can be attached to the adapter (9).

What is claimed is:

1. An OZONE, UVC LIGHT AND STEAM SANITIZER WITH ULTRASONIC NEBULIZER”, comprising:
an electro mechanical device, composed of source (1), activated by a button with LED (2), which will activate the control board (13), responsible for activating the UVC (ultraviolet germicidal) lamp (11) controller module (14), the ozone generator (10) controller module (15), and the steam generator (8), which along with water sensor (3) will vaporize the water within the water compartment (12) and the fans (4) will circulate the ozonized steam through the outlet grid (5); and
under the device are the feet (6) and the cooling grid (7), characterized by; button (2) connected to the control board (13), connected to the UVC (ultraviolet germicidal) lamp (11) controller module (14), to the ozone generator (10) controller module (15), and to the steam generator (8), which is located in the water compartment (12), which contains a sensor (3) on which the fans (4) are located under the grid (5).

2. An OZONE, UVC LIGHT AND STEAM SANITIZER WITH ULTRASONIC NEBULIZER”, according to claim 1, where an adapter (9) with two cones is coupled in the grid position (5) where parts such as gloves, boots and others will be fitted, characterized by; adapter (9).
